(19) United States
(12) Patent Application Publication
(10) Pub. No.: US 2021/0000526 A1
(43) Pub. Date: Jan. 7, 2021

(54) METHOD OF MANUFACTURING MEDICAL HEATER, MEDICAL HEATER, TREATMENT TOOL, AND TREATMENT SYSTEM

(71) Applicant: OLYMPUS CORPORATION, Tokyo (JP)

(72) Inventors: Michito MATSUOKA, Tokyo (JP); Tsunetaka AKAGANE, Tokyo (JP)

(73) Assignee: OLYMPUS CORPORATION, Tokyo (JP)

(21) Appl. No.: 17/028,218

(22) Filed: Sep. 22, 2020

Related U.S. Application Data
(63) Continuation of application No. PCT/JP2018/018308, filed on May 11, 2018.

(51) Int. Cl. A61B 18/08; H01R 43/02; B23K 11/00

(52) U.S. Cl. CPC A61B 18/082; H01R 43/0214; A61B 2018/00077; B23K 11/00; A61B 18/085

(57) ABSTRACT

A method of manufacturing a medical heater that includes arranging, on an electrically isolated substrate, (i) a heating element configured to generate heat based on a supply of electric current to the heating element, (ii) a metal film electrically connected to the heating element, and (iii) a wiring member bonded to the film. The method arranges a lead wire of the wiring member on the film, and arranges a metal bypass member on the film. A pair of welding electrodes are connected with the lead wire, and then welding the lead wire to the film occurs by allowing electric current to flow between the pair of welding electrodes.

This application is a continuation of International Application No. PCT/JP2018/018308, filed on May 11, 2018, the entire contents of which are incorporated herein by reference.

### BACKGROUND

The present disclosure relates to a method of manufacturing a medical heater, a medical heater, a treatment tool, and a treatment system.

There has been known a treatment tool used for treating a part of biological tissue to be treated (referred to as “target area”, hereinafter) with the aid of energy applied thereto.

A known treatment tool employs a medical heater (heating sheet) for applying heat energy to the target area. The medical heater has an electrically isolated substrate, a heating element (heating electrical resistor pattern) arranged on the substrate and producing heat upon current supply, and a film (heating lead connection part) arranged on the substrate and energizing the heating element. The film has a wiring member connected thereto. To the heating element, voltage is applied by way of the wiring member and the film. The heating element thus generates heat.

### SUMMARY

According to one aspect of the present disclosure, there is provided a method of manufacturing a medical heater that includes arranging, on an electrically isolated substrate, (i) a heating element configured to generate heat based on a supply of electric current to the heating element, (ii) a metal film electrically connected to the heating element, and (iii) a wiring member bonded to the film; arranging a lead wire of the wiring member on the film; arranging a metal bypass member on the film; connecting a pair of welding electrodes with the lead wire; and welding the lead wire to the film by allowing electric current to flow between the pair of welding electrodes.

According to another aspect of the present disclosure, there is provided a medical heater including an electrically isolated substrate; a heating element arranged on the substrate, the heating element being configured to generate heat based on a supply of electric current to the heating element; a metal film arranged on the substrate and electrically connected to the heating element, the film including (i) a bypass region in which a metal bypass member is arranged, and (ii) a welding region; and a wiring member including a lead wire, a part of the lead wire being welded to the film, and the part of the lead wire is welded in the welding region.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a drawing illustrating a treatment system according to a first embodiment;

FIG. 2 is a drawing illustrating a gripper;

FIG. 3 is a drawing illustrating a medical heater;

FIG. 4 is a drawing illustrating a welded part of the heating lead wire onto the film;

FIG. 5 is a flow chart illustrating a method of manufacturing the medical heater;

FIG. 6A is a drawing illustrating the method of manufacturing the medical heater;

FIG. 6B is a drawing illustrating the method of manufacturing the medical heater;

FIG. 6C is a drawing illustrating the method of manufacturing the medical heater;

FIG. 6D is a drawing illustrating the method of manufacturing the medical heater;

FIG. 7 is a circuit diagram schematically illustrating relations among a resistance welder, the film and the heating lead wire;

FIG. 8 is a drawing illustrating a welded part of the heating lead wire onto the film according to a second embodiment; and

FIG. 9 is a drawing illustrating a medical heater according to a third embodiment.

### DETAILED DESCRIPTION

Modes for carrying out the present disclosure (referred to as “embodiment”, hereinafter) will be explained, referring to the attached drawings. Note, however, that the present disclosure is by no means limited by the embodiments below. Also note that all equivalent parts in all drawings will be given the same reference signs.

### First Embodiment

Overall Structure of Treatment System

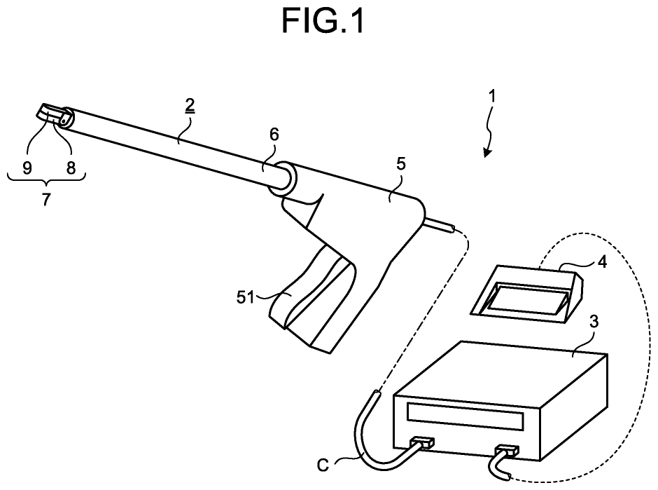

FIG. 1 is a drawing illustrating a treatment system 1 according to the first embodiment.

The treatment system 1 treats an area of biological tissue to be treated (referred to as “target area”, hereinafter) with the aid of heat energy applied thereto. Now the treatment typically means coagulation and incision of the target area. The treatment system 1 has, as illustrated in FIG. 1, a treatment tool 2, a controller 3, and a foot switch 4.

Structure of Treatment Tool

The treatment tool 2 is, for example, a surgical treatment tool used for trans-abdominally treating a target area. The treatment tool 2 has, as illustrated in FIG. 1, a handle 5, a shaft 6, and a gripper 7.

The handle 5 is a part held by an operator's hand. The handle 5 has, as illustrated in FIG. 1, an operation knob 51.

The shaft 6 has a near cylindrical shape, and is connected at one end thereof to the handle 5. The shaft 6 has the gripper 7 attached to the other end. The shaft 6 has inside thereof an open/close mechanism (not illustrated) that opens and closes first and second gripping members 8 and 9 (FIG. 1) that compose the gripper 7, in response to operation made on the operation knob 51 by the operator. Now, an electric cable C (FIG. 1) is connected at one end thereof to the controller 3, meanwhile extended inside the handle 5 and the shaft 6 so as to make the other end reach the gripper 7.

Structure of Gripper

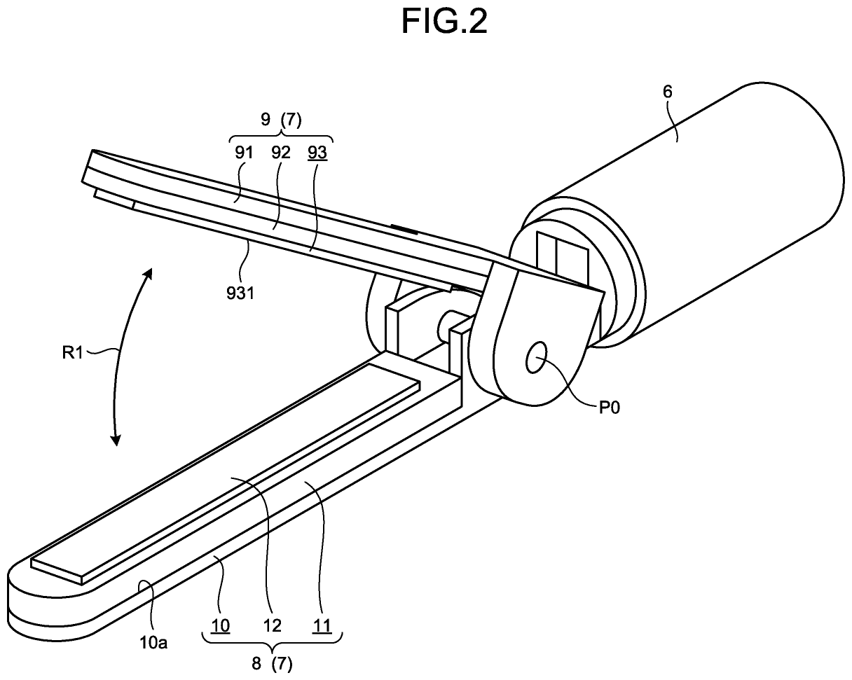

FIG. 2 is a drawing illustrating the gripper 7.

The gripper 7 is a part that treats the target area while gripping the target area. The gripper 7 has, as illustrated in FIGS. 1 and 2, the first and second gripping members 8 and 9.

The first and second gripping members 8 and 9 can open and close in the direction of arrow R1 (FIG. 2), in response to operation made on the operation knob 51 by the operator.

Structure of First Gripping Member

The first gripping member 8 is arranged at a position opposed to the second gripping member 9. The first gripping member 8 has a first jaw 10, a first heat insulating member 11, and a medical heater 12.

The first jaw 10 is a part of the shaft 6 extended therefrom towards the distal end side, and has an elongated shape that lies in the longitudinal direction connecting the distal end and the proximal end of the gripper 7. The first jaw 10 supports, on a face 10a thereof opposed to the second gripping member 9, the first heat insulating member 11 and the medical heater 12.

Materials for composing the aforementioned first jaw 10 are exemplified by metal materials such as stainless steel and titanium.

The first heat insulating member 11 is a long flat plate that extends in the longitudinal direction of the gripper 7. The first heat insulating member 11 is opposed with a ceramic substrate 121 while placing in between a heating element 124 composing the medical heater 12, and is arranged between the first jaw 10 and the medical heater 12. The first heat insulating member 11 is composed of a resin material having low heat conductivity, such as polyether ether ketone (PEEK). That is, placement of the low-heat-conductivity first heat insulating member 11, on the opposite side of the heating element 124 while placing the ceramic substrate 121 in between, enables efficient conduction of heat generated by the heating element 124 to the ceramic substrate 121.

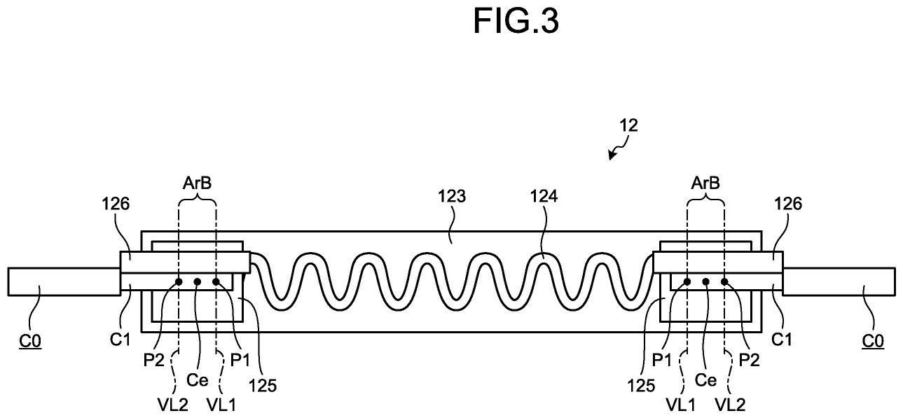

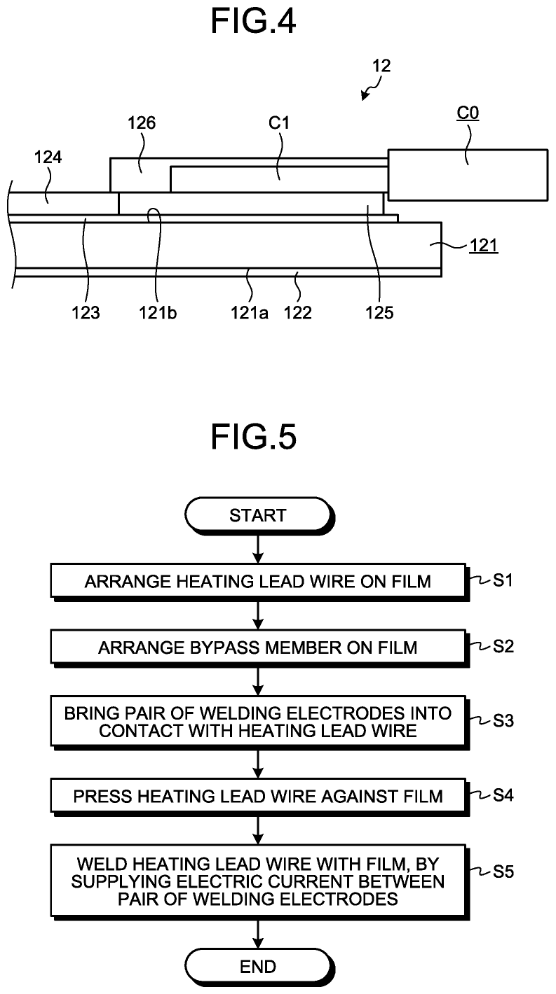

FIG. 3 is a drawing illustrating the medical heater 12. More specifically, FIG. 3 is a drawing of the medical heater 12 viewed from the side of the first heat insulating member 11. FIG. 4 is a drawing illustrating a welded part of the heating lead wire C1 onto the film 125. More specifically, FIG. 4 is a drawing illustrating the welded part viewed in the width direction orthogonal to the longitudinal direction of the medical heater 12.

The medical heater 12 is a ceramic heater that generates heat upon supply of electric current. The medical heater 12 has, as illustrated in FIG. 3 or 4, the ceramic substrate 121 (FIG. 4), an electroconductive film 122 (FIG. 4), a bonding layer 123, the heating element 124, a pair of films 125, a pair of wiring members C0 that compose the electric cable C, and a pair of bypass members 126.

The ceramic substrate 121 corresponds to the substrate in the context of the present disclosure. The ceramic substrate 121 is a long flat plate that extends in the longitudinal direction of the gripper 7, and is composed of a high-heat-conductivity ceramic material such as aluminum nitride and alumina, which are electrically insulating and heat resistant materials.

A face of the ceramic substrate 121, facing towards the second gripping member 9, serves as a first gripping face 121a (FIG. 4) that applies heat energy through the electroconductive film 122 to the target area being held between the first and second gripping members 8 and 9. Now “ . . . applies heat energy to the target area” means that heat generated by the heating element 124 is transferred to the target area.

The first gripping face 121a, although formed in a flat plane in the first embodiment, may alternatively be formed in other shapes including convex shape and concave shape. The same will apply to a second gripping face 931 described later.

The electroconductive film 122 is typically formed by metal plating, nearly over the entire range of the first gripping face 121a. The electroconductive film 122 has connected thereto a high frequency lead wire (not illustrated) composing the electric cable C.

Material composing the electroconductive film 122 is not limited to metal plating, and instead may be a mixture of a non-conductive material and a conductive material. The non-conductive material is exemplified by fluorine-containing resins such as polytetrafluoroethylene (PTFE) and perfluoroalkoxyalkane (PFA) combined with polyamide-imide (PAI); PEEK combined with silica (silicon dioxide); and PEEK combined with aerosol. Such non-conductive materials have at least one additional function such as sticking prevention against body tissue, water repellence, fat repellence, wear resistance, heat insulation, coloration and anti-halation. The electroconductive material is copper, silver or gold, for example, wherein silver is preferred.

The bonding layer 123 is typically composed of titanium oxide, and is arranged nearly over the entire range of a back face 121b (FIG. 4) of the ceramic substrate 121 on the opposite side of the first gripping face 121a. The bonding layer 123, although arranged nearly over the entire range of the back face 121b in the first embodiment, may be arranged without being limited thereto only to a part where the pair of films 125 are arranged. Material composing the bonding layer 123 is not limited to titanium oxide, instead allowing use of nickel or other material.

The heating element 124 is typically composed of tantalum, platinum or the like, and is formed on the bonding layer 123 typically by sputtering. The heating element 124 extends from one end side (right end side in FIG. 3) to the other end side (left end side in FIG. 3) of the ceramic substrate 121 in a meandering manner.

The pair of films 125 are typically composed of gold or the like, and are individually formed on the bonding layer 123 typically by sputtering. The pair of films 125 have a rectangular shape in plan view as illustrated in FIG. 3, and are individually arranged at both ends of the heating element 124 in an electrically conducting manner. Material for composing the pair of films 125 is not limited to gold, instead allowing use of stainless steel (SUS) and so forth.

The individual heating lead wires C1 composing the pair of wiring members C0 are individually welded to the pair of films 125, as illustrated in FIG. 3 or 4. To the heating element 124, voltage is applied from the controller 3, by way of the pair of heating lead wires C1 and the pair of films 125. The heating element 124 thus generates heat. Note that the heating lead wires C1 correspond to the lead wire in the context of the present disclosure.

The pair of bypass members 126 are long members individually composed of a metal material. Material for composing the pair or bypass members 126 is preferably any of those having electrical conductivity equal to or larger than electrical conductivity of the films 125. For example with the films 125 composed of gold, a possible choice is to compose the bypass members 126 with gold. Meanwhile, for example with the films 125 composed of stainless steel (SUS), a possible choice is to compose the bypass members 126 with gold, silver, copper or the like.

A method of manufacturing the aforementioned medical heater 12 will be described later.

Structure of Second Gripping Member

The second gripping member 9 has, as illustrated in FIG. 2, a second jaw 91, a second heat insulating member 92 and an opposing plate 93.

The second jaw 91 has a long shape that extends in the longitudinal direction of the gripper 7. The second jaw 91 is pivotally supported at the proximal end thereof by the shaft 6, so as to be pivotable around a fulcrum P0 (FIG. 2), wherein pivoting enables opening and closure in cooperation with the first gripping member 8.

Although the first embodiment exemplifies a structure in which the first gripping member 8 (first jaw 10) is fixed to the shaft 6, and the second gripping member 9 (second jaw 91) is pivotally supported by the shaft 6, the structure is not limited thereto. For example, both of the first and second gripping members 8 and 9 may be pivotally supported by the shaft 6, and may be allowed to open and close as a result of their pivoting. Alternatively in another employable structure, the first gripping member 8 may be pivotally supported by the shaft 6, the second gripping member 9 may be fixed to the shaft 6, and pivoting of the first gripping member 8 may enable opening and closure in cooperation with the second gripping member 9.

The second heat insulating member 92 is typically composed of a low-heat-conductivity resin material such as PEEK, and is arranged between the second jaw 91 and the opposing plate 93.

The opposing plate 93 is composed of an electroconductive material, and is fixed on a face, opposing to the first gripping member 8, of the second heat insulating member 92.

The face, opposing to the first gripping member 8, of the opposing plate 93 functions as a second gripping face 931 that grips, in cooperation with the first gripping member 8, the target area in between. The opposing plate 93 has connected thereto a high frequency lead wire (not illustrated) composing the electric cable C. By way of a pair of high frequency lead wires (not illustrated), high frequency current is supplied between the electroconductive film 122 and the opposing plate 93. In other words, the electroconductive film 122 and the opposing plate 93 individually function as high frequency electrodes.

Structure of Controller and Foot Switch

A foot switch 4 is a component operated with a foot of the operator. In response to an operation made on the foot switch 4, control over treatment is effected by the controller 3.

A means for effecting such control over treatment is not limited to the foot switch 4, instead allowing use of other switch operated by a hand.

The controller 3 has a central processing unit (CPU) or the like, and effects control over treatment in the treatment of the target area, making the treatment tool 2 operate according to a predetermined program product.

Operations of Treatment System

Next, operations of the aforementioned treatment system 1 will be explained below.

The operator holds the treatment tool 2 in hand, and inserts the distal end (the gripper 7 and a part of the shaft 6) of the treatment tool 2, for example, through an abdominal wall into an abdominal cavity, typically using a catheter. The operator then operates the operation knob 51 so as to grip the target area with the gripper 7. The operator then operates the foot switch 4. The controller 3 then controls the treatment described below.

The controller 3 supplies high frequency current between the electroconductive film 122 and the opposing plate 93, by way of a pair of high frequency lead wires (not illustrated). The target area thus generates Joule heat as a result of flow of high frequency current. Almost concurrently with the supply of high frequency current between the electroconductive film 122 and the opposing plate 93, the controller 3 also applies voltage to the heating element 124, by way of the pair of heating lead wires C1 and the pair of films 125. The controller 3 measures resistivity of the heating element 124, using values of current and voltage applied to the heating element 124, typically according to the voltage drop method. The controller 3 then varies electric power to be supplied to the heating element 124, so as to make the resistivity reach a target resistivity value, in other words, so as to carry out control for adjusting the temperature of the heating element 124 to a target temperature. That is, heat from the heating element 124 controlled at the target temperature is transferred to the target area, by way of the ceramic substrate 121.

As a result of such control for treatment, the target area is incised while being coagulated.

Method of Manufacturing Medical Heater

Next, a method of manufacturing the aforementioned medical heater 12 will be described.

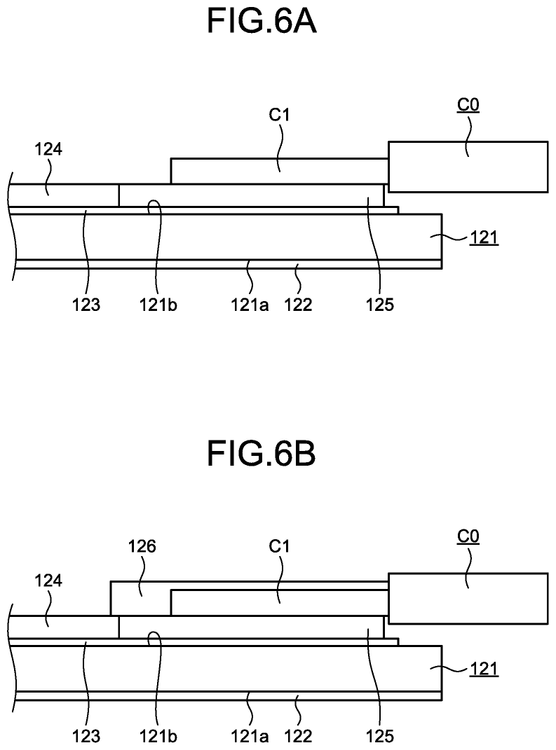

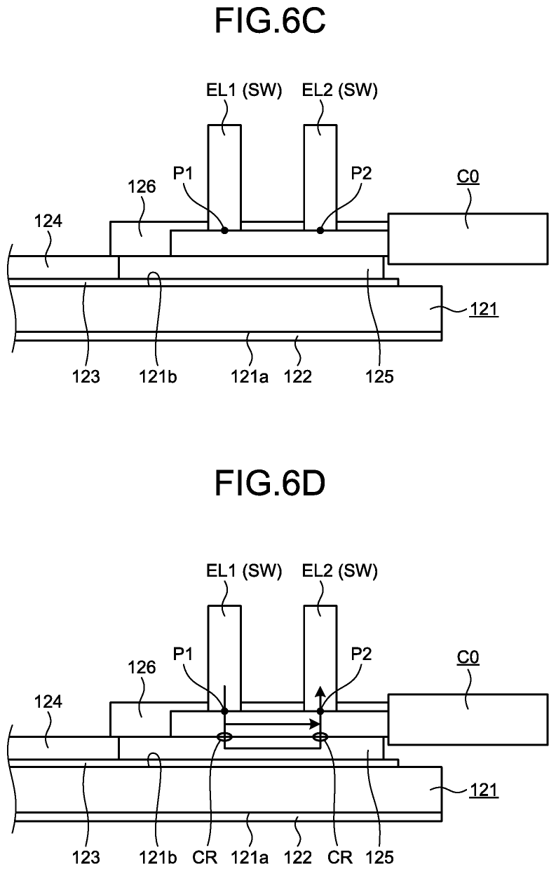

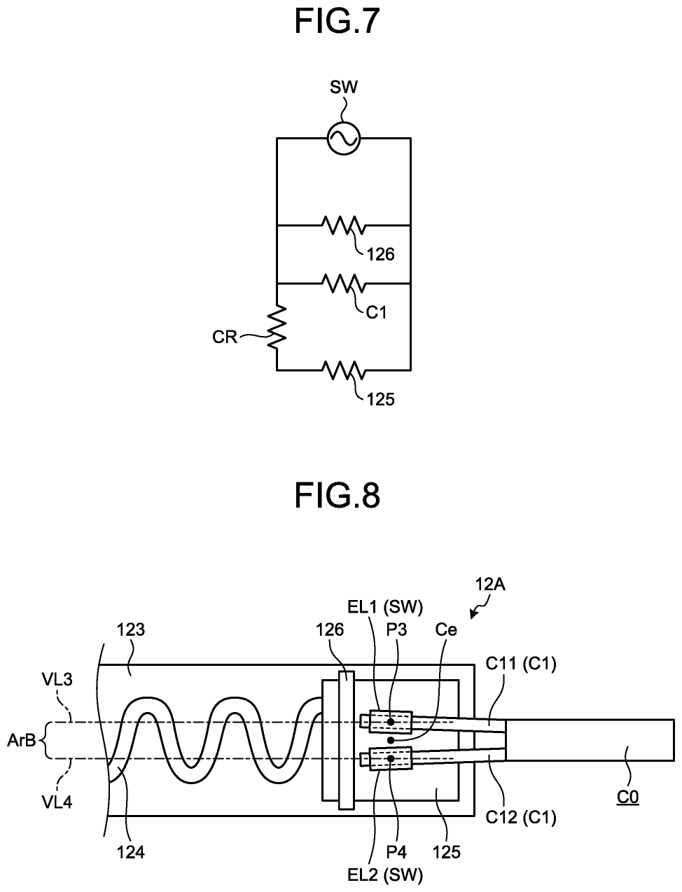

FIG. 5 is a flow chart illustrating a method of manufacturing the medical heater 12. FIGS. 6A to 6D are drawings illustrating the method of manufacturing the medical heater 12. More specifically, FIGS. 6A to 6D are drawings corresponded to FIG. 4. FIG. 7 is a circuit diagram schematically illustrating relations among a resistance welder SW, the film 125 and the heating lead wire C1.

The paragraphs below will describe the method of the manufacturing the medical heater 12, highlighting a method of welding the heating lead wire C1 to the film 125.

First, as illustrated in FIG. 6A, the operator arrange the heating lead wire C1 onto the film 125 (Step S1). More specifically, the operator in Step S1 arranges the heating lead wire C1 on the film 125, so that the distal end of the heating lead wire C1 is directed towards the heating element 124, so that the longitudinal direction of the heating lead wire C1 is aligned nearly in parallel to the longitudinal direction of the ceramic substrate 121, and so that the heating lead wire C1 lies across a center position Ce of the film 125 (FIG. 3).

After Step S1, the operator arranges as illustrated in FIG. 6B a bypass member 126 on the film 125 (Step S2). Referring now to the film 125 in plan view, two points P1, P2 (FIG. 3) are defined on the heating lead wire C1 while placing the center position Ce in between. The individual points P1, P2 correspond to the contact points in the context of the present disclosure. Now a bypass region ArB (FIG. 3) is defined by a region that falls between two virtual lines VL1, VL2 (FIG. 3) that partition the whole region of the film 125, the virtual lines VL1, VL2 being assumed to individually pass through the points P1, P2, and to lie orthogonally to the direction of connecting the points P1, P2. The operator in Step S2 arranges the bypass member 126 on the film 125, so as to place it in the bypass region ArB. The bypass member 126 has a length equal to or longer than the linear dimension between the two virtual lines VL1, VL2, or the linear dimension between the individual points P1, P2, and is arranged in the bypass area ArB so as to be laid across the two virtual lines VL1, VL2. Note that the bypass member 126 may be arranged in Step S2 in contact with the heating lead wire C1, or may alternatively be arranged not in contact with the heating lead wire C1.

After Step S2, the operator brings, as illustrated in FIG. 6C, a pair of welding electrodes EL1, EL2 of the resistance welder SW into contact with the heating lead wire C1 respectively at the points P1, P2 (Step S3).

After Step S3, the operator moves the pair of welding electrodes EL1, EL2 downwards in FIG. 6D to press the heating lead wire C1 against the film 125 (Step S4), and allows current to flow between the pair of welding electrodes EL1, EL2 (Step S5). In this process, Joule heat generates at the interface between the heating lead wire C1 and the film 125, where contact resistance CR (FIGS. 6D and 7) occurs. The heating lead wire C1 and the film 125 are mutually fused by Joule heat, and are bonded. Note that areas in the film 125 where the contact resistance CR occurs correspond to the welding region in the context of the present disclosure.

The first embodiment explained above demonstrates the effects below.

The method of manufacturing the medical heater 12 of the first embodiment employs Step S2 in which the bypass member 126 made of metal is arranged on the film 125, before Step S5 in which the heating lead wire C1 and the film 125 are welded by allowing current to flow between the pair of welding electrodes EL1, EL2. In Step S2, the bypass member 126 is eventually arranged, as illustrated in FIG. 7, in parallel with the film 125 and the heating lead wire C1, with respect to the resistance welder SW. The bypass member 126 has electrical conductivity equal to or larger than that of the film 125. Hence, even if overcurrent should flow in Step S5, due to resistivity of the contact resistance CR of an article to be welded, that are the film 125 and the heating lead wire C1, most of such overcurrent can be bypassed through the bypass member 126, successfully reducing value of current that flows through the article to be welded. In other words, this successfully makes the article to be welded less susceptible to damage possibly caused by the overcurrent.

The first embodiment thus demonstrates an effect of enhancing bonding strength between the film 125 and the heating lead wire C1.

With the bonding strength between the film 125 and the heating lead wire C1 thus stabilized, it now becomes possible to accurately measure resistivity of the heating element 124, and to accurately adjust the heating element 124 to a target temperature, on the basis of such resistivity.

In particular in the first embodiment, the bypass member 126 in Step S2 is arranged on the film 125, so as to be laid in the bypass region ArB. The bypass member 126 has a length equal to or longer than the linear dimension between the two virtual lines VL1, VL2, and is arranged in the bypass area ArB so as to be laid across the two virtual lines VL1, VL2.

Hence, most of the aforementioned overcurrent can be bypassed more effectively to the bypass member 126, as compared with an exemplary case in which the bypass member 126 were arranged on the film 125 but outside the bypass region ArB, or as compared with a case where the bypass member 126 were arranged on the film 125 but with the longitudinal direction of the bypass member 126 aligned in parallel to the two virtual lines VL1, VL2 in the bypass region ArB. In short, the article to be welded can effectively be made less susceptible to damage possibly caused by the overcurrent.

In the first embodiment, the film 125 is a gold film.

Thus the film 125 will have a surface condition more largely stabilized as compared with the film 125 composed of other material. In other words, the contact resistance CR will be suppressed from varying for every article to be welded.

In the first embodiment, the ceramic substrate 121 is used as the substrate in the context of the present disclosure.

Hence, the ceramic substrate 121 will be functionalized as a heat transfer plate through which heat from the heating element 124 is transferred to the target area. This means there is no need to separately provide a heat transfer plate, making it possible to reduce the number of components of the medical heater 12, and to downsize the medical heater 12.

In the first embodiment, the bonding layer 123 is arranged between the ceramic substrate 121 and the film 125.

Hence, the bonding strength between the ceramic substrate 121 and the film 125 can be improved, with the aid of the bonding layer 123.

### Second Embodiment

Next, a second embodiment will be explained.

In the explanation below, all structures similar to those in the aforementioned first embodiment will be given the same reference signs, in order to skip or simplify the detailed explanation.

The second embodiment is different from the aforementioned the first embodiment, in a method of welding the heating lead wire C1 to the film 125 (the method of manufacturing the medical heater 12 according to the present disclosure).

FIG. 8 is a drawing illustrating a welded part of the heating lead wire C1 onto the film 125 according to the second embodiment. More specifically, FIG. 8 is a drawing of the welded part viewed from the side of the first heat insulating member 11.

The method of manufacturing a medical heater 12A of the second embodiment will be explained, referring to FIGS. 5 and 8.

First, the operator branches the heating lead wire C1 into two strands, which are a first lead wire C11 and a second lead wire C12, as illustrated in FIG. 8. The operator then arranges the heating lead wire C1 on the film 125 (Step S1). More specifically, the operator in Step S1 arranges the first and second lead wires C11, C12 on the film 125, so that the distal ends of the first and second lead wires C11, C12 are directed towards the heating element 124, while placing the center position Ce between the first and second lead wires C11, C12.

After Step S1, the operator arranges as illustrated in FIG. 8 the bypass member 126 on the film 125 (Step S2). Referring now to the film 125 in plan view, two points P3, P4 (FIG. 8) are respectively defined on the first and second lead wires C11, C12 while placing the center position Ce in between. The individual points P3, P4 correspond to the contact points in the context of the present disclosure. Now the bypass region ArB (FIG. 8) is defined by a region that falls between two virtual lines VL3, VL4 (FIG. 8) that partition the whole region of the film 125, the virtual lines VL3, VL4 being assumed to individually pass through the points P3, P4, and to lie orthogonally to the direction of connecting the points P3, P4. The operator in Step S2 arranges the bypass member 126 on the film 125, so as to place it in the bypass region ArB. The bypass member 126 has a length equal to or longer than the linear dimension between the two virtual lines VL3, VL4, in other words, the linear dimension between the individual points P3, P4, and is arranged in the bypass area ArB so as to be laid across the two virtual lines VL3, VL4. Note that the bypass member 126 may be arranged in Step S2 in contact with the heating lead wire C1, or may alternatively be arranged not in contact with the heating lead wire C1.

After Step S2, the operator brings, as illustrated in FIG. 8, the pair of welding electrodes EL1, EL2 of the resistance welder SW into contact with the first and second lead wires C11, C12 respectively at the points P3, P4 (Step S3).

After Step S3, the operator moves the pair of welding electrodes EL1, EL2 to press the first and second lead wires C11, C12 against the film 125 (Step S4), and allows current to flow between the pair of welding electrodes EL1, EL2 (Step S5). In this process, Joule heat generates at the interface between the first and second lead wires C11, C12 and the film 125 where contact resistance CR (not illustrated) occurs. The first and second lead wires C11, C12 and the film 125 are mutually fused by Joule heat, and are bonded.

The second embodiment explained above demonstrates the effects below, besides the effects comparable to those of the first embodiment.

In a method of manufacturing the medical heater 12A of the second embodiment, the heating lead wire C1 branched into two strands is arranged on the film 125 in Step S2. In Step S3, the pair of welding electrodes EL1, EL2 of the resistance welder SW are brought into contact with the first and second lead wires C11, C12 respectively at the points P3, P4. Then in Step S5, current is allowed to flow between the pair of welding electrodes EL1, EL2, to thereby weld the first and second lead wires C11, C12 with the film 125.

Hence, even if overcurrent should flow in the article to be welded in Step S5 to unfortunately break the film 125 so as to isolate the points P3, P4, the first lead wire C11 and the heating element 124 can remain electrically connected by way of either one fragment of the broken film 125. That is, the heating element 124 can remain energized by way of either one fragment of the film 125 and either one strand of the first lead wire C11.

### Third Embodiment

Next, a third embodiment will be explained.

In the explanation below, all structures similar to those in the aforementioned first embodiment will be given the same reference signs, in order to skip or simplify the detailed explanation.

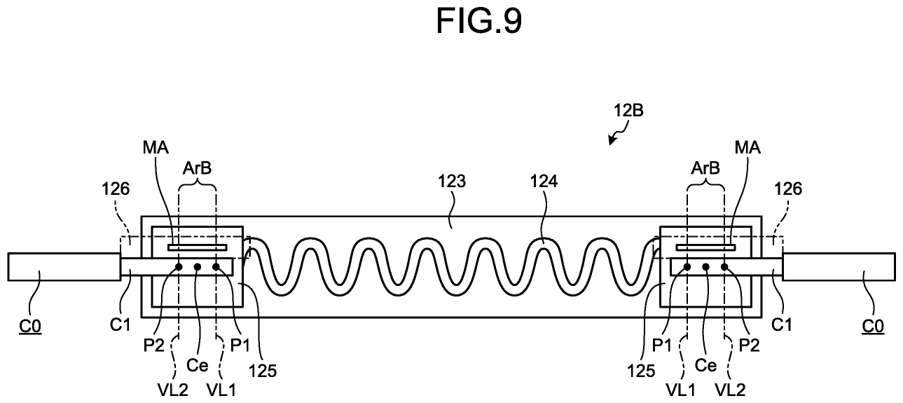

FIG. 9 is a drawing illustrating a medical heater 12B according to the third embodiment. More specifically, FIG. 9 corresponds to FIG. 3.

The medical heater 12B of the third embodiment, as illustrated in FIG. 9, does not have the pair of bypass members 126, unlike the medical heater 12 explained in the first embodiment.

More specifically, the medical heater 12B is obtainable by the aforementioned method of manufacturing the medical heater 12 explained in the first embodiment, but by removing the bypass member 126 after the heating lead wire C1 was welded to the film 125. Hence the film 125 has a mark MA remained thereon across the bypass area ArB after removal of the bypass member 126, as illustrated in FIG. 9.

Also the aforementioned third embodiment, even with the bypass member 126 removed after welding the heating lead wire C1 to the film 125, can demonstrate the effects comparable to those in the first embodiment.

### Other Embodiment

Having described Embodiments for carrying out the present disclosure, the present disclosure is by no means limited by aforementioned first to third embodiments.

The medical heaters (12, 12A, 12B) of the present disclosure, although being ceramic heaters in aforementioned first to third embodiments, are not limited thereto. For example, employable as the medical heater of the present disclosure may be a sheet heater having a sheet-like substrate composed of an electrical insulating material such as polyimide, and the heating element and the film employed by the present disclosure arranged thereon.

In aforementioned first to third embodiments, the shape of the heating element 124 is not limited to the shape explained in first to third embodiments, but may typically be a U-shape conforming to the outer contour of the ceramic substrate 121.

In aforementioned first to third embodiments, the bypass member 126 may be arranged in a posture not limited to that explained in first to third embodiments, and may be arranged in any posture if only it were not in parallel with the virtual lines VL1, VL2 (VL3, VL4).

The process flow representing the method of manufacturing the medical heater 12 (12A, 12B) does not always necessarily conform to the order of steps in the flow chart (FIG. 5) explained in first to third embodiments, and may be modified without causing contradiction. For example, Steps S1 and S2 may be carried out in the inverted order.

The medical heater 12 (12A, 12B), although being arranged only on the first gripping member 8 in aforementioned first to third embodiments, is not limited thereto. The medical heater 12 (12A, 12B) may be arranged on both of the first and second gripping members 8 and 9.

According to the method of manufacturing a medical heater, a medical heater, a treatment tool and a treatment system of the present disclosure, it now becomes possible to enhance bonding strength between the film and the lead wire.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A method of manufacturing a medical heater, the method comprising:
arranging, on an electrically isolated substrate, (i) a heating element configured to generate heat based on a supply of electric current to the heating element, (ii) a metal film electrically connected to the heating element, and (iii) a wiring member bonded to the film;
arranging a lead wire of the wiring member on the film;
arranging a metal bypass member on the film;
connecting a pair of welding electrodes with the lead wire; and
welding the lead wire to the film by allowing electric current to flow between the pair of welding electrodes.

2. The method according to claim 1, further comprising pressing the lead wire against the film.

3. The method according to claim 1, wherein:
a bypass region is formed on the film, and
the bypass member is arranged such that at least a part of the bypass member is located in the bypass region.

4. The method according to claim 3, wherein the bypass region is a region that is located between two virtual lines that partition a region covering all of the film, the virtual lines individually passing through contact points of the pair of welding electrodes to the lead wire, and the virtual lines are located orthogonally to a direction connecting the contact points.

5. The method according to claim 4, wherein:
the bypass member is formed into an elongated shape with a linear dimension equal to or greater than a linear dimension between the two virtual lines, and
the bypass member is arranged to cross the two virtual lines.

6. The method according to claim 1, wherein:
the lead wire branches into a first strand and a second strand,
one welding electrode of the pair of welding electrodes contacts the first strand of the lead wire, and the other welding electrode of the pair of welding electrodes contacts the second strand of the lead wire, and
both of first strand and the second strand of the lead wire are individually welded to the film.

7. The method according to claim 1, wherein the film is formed of gold.

8. The method according to claim 1, wherein the bypass member has electrical conductivity equal to or greater than electrical conductivity of the film.

9. The method according to claim 1, wherein the substrate is formed of ceramic.

10. A medical heater comprising:
an electrically isolated substrate;
a heating element arranged on the substrate, the heating element being configured to generate heat based on a supply of electric current to the heating element;
a metal film arranged on the substrate and electrically connected to the heating element, the film including (i) a bypass region in which a metal bypass member is arranged, and (ii) a welding region; and
a wiring member including a lead wire, a part of the lead wire being welded to the film, and the part of the lead wire is welded in the welding region.

11. The medical heater according to claim 10, wherein the bypass member is arranged such that at least a part of the bypass member is located in the bypass region.

12. The medical heater according to claim 11, wherein the bypass region is located between two virtual lines that partition a region covering all of the film, the virtual lines individually passing through contact points of a pair of welding electrodes contacting the lead wire when the film and the lead wire is welded, and the virtual lines are arranged orthogonally to a direction connecting the contact points.

13. The medical heater according to claim 12, wherein the bypass member is formed into an elongated shape with a linear dimension equal to or greater than a linear dimension between the two virtual lines, and the bypass member is arranged so as to cross the two virtual lines.

14. The medical heater according to claim 11, wherein the bypass member has electrical conductivity equal to or greater than electrical conductivity of the film.

15. The medical heater according to claim 10, wherein the lead wire branches into a first lead wire and a second lead wire and each are individually welded to the film.

16. The medical heater according to claim 10, wherein a mark is formed in the bypass region after removal of the bypass member.

17. The medical heater according to claim 10, wherein the film is formed of gold.

18. The medical heater according to claim 10, wherein the substrate is formed of ceramic.

19. The medical heater according to claim 10, further comprising a bonding layer arranged between the substrate and the film that bonds the substrate and the film.

20. A treatment tool comprising the medical heater according to claim 10.
